(19) United States
(12) Patent Application Publication
(10) Pub. No.: US 2017/0146450 A1
(43) Pub. Date: May 25, 2017

(54) SPECIES SPECIFIC SENSOR FOR EXHAUST GASES AND METHOD THEREOF

(71) Applicant: Sentelligence, Inc., Carmel, IN (US)

(72) Inventor: John Coates, Newtown, CT (US)

(21) Appl. No.: 15/357,014

(22) Filed: Nov. 21, 2016

Related U.S. Application Data
(60) Provisional application No. 62/257,507, filed on Nov. 19, 2015.

(51) Int. Cl. G01N 21/33; F01N 13/00; F01N 11/00; G01N 33/00; F01N 3/28

(52) U.S. Cl. CPC G01N 21/33; G01N 33/0029; G01N 33/0037; G01N 33/0054; G01N 33/0042; F01N 3/2882; F01N 11/007; F01N 13/008; G01N 2201/061; G01N 2201/08; G01N 2201/062; F01N 2560/026; F01N 2560/027; F01N 2560/021

(57) ABSTRACT

A species-specific gas sensor and monitor comprising a light source, a sample enclosure or measurement chamber, an optical interface between the light source, the sample and the detection system, electronics that integrate the light source and the detection system, and computational components, such as an onboard microprocessor for calculation of the gas composition and communications between the sensor and the vehicle electronics. The species-specific gas sensor of the present invention can be used to target gases, such as nitric oxide (NO), nitrogen dioxide (NO₂) ammonia (NH₃), and sulfur dioxide (SO₂) which are measurable in the UV spectrum.

### CROSS-REFERENCE TO RELATED APPLICATION

This U.S. patent application claims priority to U.S. Provisional Application 62/257,507 filed Nov. 19, 2015, the disclosure of which is considered part of the disclosure of this application and is hereby incorporated by reference in its entirety.

### FIELD OF THE INVENTION

This invention relates generally to ultraviolet (UV)/visible spectroscopy of gas phase mixtures. In one aspect, the present invention relates to species-specific detectors to detect and monitor the levels of individual gas species.

### BACKGROUND

The analytical spectral region for exhaust gases extends from the UV to the mid infrared (mid-IR). Because of this, in many industries, and in particular the automotive industry, infrared and UV gas analyzers are used to continuously measure the real-time concentration of each component in a gas sample that contains various gas components by selectively detecting the amounts of infrared radiation absorbed by the gas components. The infrared gas analyzer is widely used in various fields because of its excellent selectivity and a high measuring sensitivity. Non-dispersive infrared (NDIR) techniques for the analysis of exhaust gases for individual species monitoring are a common approach used for an infrared gas analyzer. NDIR instruments use filters to isolate the wavelengths relevant to the specific gases being detected. Commercial systems based on UV absorption can be used for combustion gas emissions monitoring in the power generation and industrial combustion processes. These systems use commercial spectrometers for the measurements, which can be large in size and very expensive.

Single-beam and two-beam NDIR gas analyzers are known. With single-beam devices, the infrared radiation generated by the infrared emitter is routed after modulation, such as by a rotating filter wheel, through the measuring vessel containing the gas mixture with the measuring gas component to the detector device. With two-beam devices, the infrared radiation is subdivided into a modulated measuring radiation passing through the measuring vessel and into an inversely-phased modulated comparison radiation passing through a comparison vessel filled with a comparison or reference gas. Alternatively, the second beam can perform as an optical reference to light source compensation. Opto-pneumatic detectors have been used as the preferred detector device. These detectors are filled with the gas components to be verified and comprise one or more receiver chambers arranged adjacent or to the rear of one another. Such devices are used in a signal handling approach known as gas filter correlation measurements.

Other spectroscopy methods used in monitoring fluids include those disclosed in U.S. Pat. No. 7,339,657 to Coates et al., which is incorporated herein by reference. These examples feature near infrared LEDs that are used for oil condition measurements (soot level) and urea in selective catalytic reduction (SCR) fluids, such as the diesel exhaust fluid (DEF) AdBlue®. The soot measurement is a simple photometric measurement with one primary wavelength (940 nm), while the urea-quality sensor is a true spectral measurement with a three-point determination having two analytical wavelengths, 970 nm and 1050 nm, for water and urea, and one as reference/baseline, 810 nm. In both cases, attenuation of signal intensity is used to compute the infrared (near-infrared) absorption, and this is correlated to the concentrations of soot (in oil) and the relative concentrations of water and urea in the binary mixture/solution.

In addition to the NDIR gas analyzer above, a second approach to monitoring exhaust gases is through the use of probes and Light Emitting Diode (LED)-based sensors using UV absorption. Many of the exhaust gases desired to be measured for emissions monitoring fall within the UV and visible spectrum. In the UV exhaust monitoring platform, the LEDs are used to define the wavelengths that are used for making the spectral measurements. The two main mono-nitrogen oxides (NOx) gases, nitric oxide (NO) and nitrogen dioxide (NO₂), have characteristic absorption spectra in the UV and deep UV spectral regions, and NO₂ partially in the visible.

Only NO₂ absorbs in the UV and the visible, and both gases absorb in the deep UV (between 200 nm and 250 nm). The application of using deep UV for monitoring has expanded beyond just NOx, to further include ammonia gas. Ammonia can come in the form of liquid ammonia or a decomposition product from a near saturated solution of urea (32.5% urea in water). In this latter case, the catalytic decomposition of urea by a technique known as selective catalytic reduction (SCR) yields ammonia gas, which reacts with NOx species in the presence of the catalyst material to neutralize them. While the SCR reaction has the desired effect of removing the NOx, a secondary issue is the potential release of excess ammonia gas, a condition known as ammonia slip. As a result, many sensor systems are required to measure ammonia as well as the NOx, and this can be accomplished in the deep UV at wavelengths between 200 nm and 225 nm.

Finally, one important class of gas contaminants that can be present in diesel engine exhaust are sulfur oxides, and in particular sulfur dioxide. Although this is a separate measurement and is not presently subject to environmental regulation, it is a practical issue, especially when low-grade fuels are obtained from regions having high sulfur levels. The addition of sulfur dioxide as one of the measurement gases is capable of being monitored with UV sensors because sulfur dioxide has UV absorption between the two absorption bands of nitrogen dioxide. Therefore, to complete the measurement suite, the final fuel monitoring system can be configured to measure the three gases (NO, NO₂ and NH₃) in real time, as well as SO₂ for the assessment of sulfur. All of these gases can be measured on commercial gas analyzer systems for NOx reduction and emissions control measurements of combustion gases. Systems featuring a small spectrometer configured for the deep UV (down to 200 nm) are available to the Continuous Emissions Monitoring (CEM) market, the smoke stack monitoring market and the automotive emissions control market.

LED components are available that support an extended spectral region from the UV region to around 250 nm and mid-IR into about the 3 to 5 micron region. These devices are currently expensive and do not have a good usable lifetime in the context of low-cost automotive sensors. Both of these LED regions are important for the application to exhaust gas sensing. The mid-infrared region is established for exhaust gas monitoring primarily combustion gases, CO and CO₂, and to some extent NOx and other pollutant gases.

However, prior LED sensing platforms are not reliable for high temperature gas monitoring, and the implementation relative to the optics required is difficult, if not impossible. While using an NDIR concept as a dedicated sensor is feasible, it is not commercially practical because of the need for a long physical optical path required for IR detection. Further, major combustion gas components, such as carbon dioxide (CO₂), carbon monoxide (CO) and water are all infrared absorbers. Water in particular can become a matrix interferent and prevent accurate readings.

The infrared and ultraviolet systems described above are designed as high-end analyzer systems for the process, industrial and environmental markets. In their commercially available forms they are not adaptable as a low-cost inline or in situ sensing system for the diesel engine market. Additionally, a dirty gas stream such as diesel engine exhaust presents a challenge when constructing a gas sensor. The fine particulate from soot has a tendency to penetrate small areas and potentially attenuate optical beams on reflective surfaces. In addition, crosstalk may occur between components of the gas sensor system. Finally, the high temperatures and wide range of operating temperatures demand close attention to the construction and construction materials used for the optical interface. There exists a need for a low cost, species-specific sensor for the analysis of diesel exhaust gases using deep UV to provide the ability to measure the species NO, NO₂, SO₂, NH₃, and certain Aromatics (Ar) in overcoming the aforementioned obstacles.

### BRIEF SUMMARY OF THE INVENTION

In one aspect, this disclosure is related to a species specific gas sensor and monitor comprising a light source, a sample enclosure or measurement chamber having an opening for said sample, an optical interface between the light source the sample and the detection system, an optical interface between the light source and the sample measurement chamber, a detector module, electronics configured to integrate the light source and the detection system, and computational components, such as an onboard microprocessor for calculation of the gas composition and communications between the sensor and the vehicle electronics.

In another aspect, this disclosure is related to an implementation of the present invention involving replacing a spectrometer by a dedicated single or multiple wavelength detector made from a combination of a UV sensitive detector(s) and a dedicated close-coupled filter intimately placed on surface of the detector. In one exemplary embodiment of the present invention the detector is fabricated with a detector material coated directly on top of the surface of the detector.

In yet another aspect, this disclosure is related to a real-time measurement sensor of NOx gas species using solid state light source, such as an LED, and a solid state detector package, such as a standard photodiode detectors for detection. This sensor can be based on a 360 nm or 400 nm LED for NO₂ and a 700 nm LED for a reference baseline. This implementation can also be implemented with a remote insertion probe, or the LED light sources may be mounted outside the sensor enclosure and close coupled a measurement chamber having a quartz or fused silica light guide. The sensor uses a coupling apparatus for coupling said solid-state source and solid-state detector to the measurement chamber. The measurement chamber may also include a single component optical interface fabricated as a refractive optic that works in an internal reflectance or optional transmittance modes (or light scattering or fluorescence modes). Integrated electronics that include circuits that provide optical compensation, temperature sensing and compensation, analog and digital signal processing, and external communications are communicatively coupled to the sensor. The system is designed to allow a high level of integration of both electronic and optical components, and to include packaging that provides both thermal isolation and ease of assembly and manufacture. Fiber optics or other forms of optical light guide or light conduit may be used, with appropriate source collimation and detector collection optical elements.

### BRIEF DESCRIPTION OF THE DRAWINGS

The features and advantages of this disclosure, and the manner of attaining them, will be more apparent and better understood by reference to the following descriptions of the disclosed system and process, taken in conjunction with the accompanying drawings, wherein:

FIG. 1 is a view of an exemplary embodiment of a species specific gas sensor.

FIG. 2A is a cross section view of exemplary embodiment of a fiber-optic coupled insertion style gas sensing probe.

FIG. 2B is a bottom view of exemplary embodiment of a fiber-optic coupled insertion style gas sensing probe.

FIG. 3A is a cross-section illustration of an exemplary embodiment of a LED-based sensor platform for gas and vapor measurements.

FIG. 3B is an illustration of an exemplary embodiment of an opto-board for the sensor shown in FIG. 3A.

FIG. 3C is an illustration of an exemplary embodiment of an optical isolator for the sensor shown in FIG. 3A.

FIG. 4A is a perspective view of an exemplary embodiment of a 50 millimeter measurement chamber made from aluminum.

FIG. 4B is a perspective view of an exemplary embodiment of a 100 millimeter measurement chamber made from stainless steel.

FIG. 5A is a gas phase UV spectra for NO and NO₂.

FIG. 5B is a gas phase UV spectra for NO, NO₂, and ammonia.

FIG. 6A is gas phase UV spectra for NO.

FIG. 6B is gas phase UV spectra for NO₂.

FIG. 6C is gas phase UV spectra for ammonia.

### DETAILED DESCRIPTION OF THE INVENTION

The present invention relates to a species specific gas sensor having a measurement range from deep-UV (100 nm) to visible (vis) light spectrum (750 nm). The species-specific gas sensor of the present invention can be used to target gases, such as nitric oxide (NO), nitrogen dioxide (NO₂) ammonia (NH₃), and sulfur dioxide (SO₂) which are measurable in the UV spectrum.

One preferred embodiment of the sensor is a low voltage device having minimal power requirement. The device may be made available with various electronics packages, from a simple digital output device to a smart sensor that provides processed numerical data. The output from the sensor can either go directly to a display, such as a simple status light or to an alpha-numeric or a graphical display. For example, the status light may be a three-state LED: green (OK), yellow (warning) and red (alert or problem), and the graphical display may be an LCD display. Alternatively, the sensor can provide a standard format output to a vehicle or equipment data bus that supplies diagnostic data to an on-board computer, which in turn supports an intelligent sensor output display.

With one or more of the optical sensors on board a vehicle, there is the need to provide the results back via some form of a display or on-board data handling system. Most heavy-duty vehicles already have a significant number of sensor systems in place that communicate back to the operator/driver via alarms, alerts, displays or status lights. In some cases, these are activated by direct connections with the sensor or via a vehicle management system involving an on-board computer and data manager. The present invention can be installed by an OEM during the manufacturing process of the vehicle or engine where it can be integrated into a vehicle management system. Alternatively, the sensor can be an aftermarket component that can be integrated using a direct connection route to a simple status display on the dashboard.

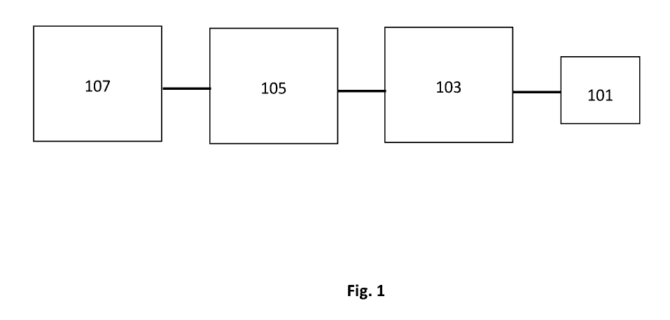

While other embodiments exist, two primary embodiments of the sensor can be implemented to provide a desired real-time measurement of the NOx gas species in an exhaust gas measurement system. One exemplary embodiment, shown in FIG. 1, can include an in-line sensor that utilizes a flow-through sample chamber 101 or interface. The flow through chamber method of measurement is compatible with a fiber optic arrangement defined below. It can be implemented in a bypass arrangement where the hot gas stream is diverted and passed through the flow chamber, after which it may be emitted as exhaust or returned to the exhaust gas flow. In the in-line embodiment of the sensor, the temperature of the exhaust gases can be reduced down to a range between ambient and about 120° C. by dilution of the exhaust gas with cold air. Lower temperature allows for flexibility in terms of the placement of the optics and the measurement electronics, including close coupling that would allow for compact assembly of the sensor.

Referring still to FIG. 1, the sensor can use a suitable lamp 103 that delivers radiation over the full UV-vis operational wavelength range, such as from about 195 nm to about 700 nm. For example, a deuterium discharge lamp or a xenon flash lamp are well suited for providing deep UV, as well as longer UV wavelengths. Additionally, for applications extending further to, e.g., about 700 nm in the visible light spectrum, a pulsed xenon lamp may be a preferred option for a low-cost light source. Wavelength selection can be made using a narrow bandpass filter, among other types of wavelength filtering methods. The filter can either be a separate filter that is placed on top of the detector or may be fabricated as a filter material coated directly on top of the detector surface.

The present invention uses an approach to detection and monitoring gas samples distinct from typical approaches for exhaust monitoring systems that use a miniature spectrometer as the detection system Specifically, the example shown in FIG. 1 is a fully integrated system containing the light source 103, a spectrometer 105 for detection, measurement and acquisition, and control and communication electronics 107. Another exemplary embodiment of the present invention can have a custom detector system that can feature a composite detector with multiple detection elements.

Each detection element can be optimized for the wavelength of the specific gas components or variables desired to be measured. The number of detectors integrated will depend on the optimum number of variables or gas species to be measured. The individual detector elements are selected based on the optimum choice for detecting the selected wavelength. The wavelength selection is also part of each detector assembly and can be provided by a custom light filtration system that can be physically combined with each detector element. The detector electronics are optimally integrated with the detector elements, and the operation of the detector is synchronized to the light source or source modulation, such as a pulsed xenon source.

The source and detection system are coupled to electronic systems that optimize the collection of the optical/spectral data. The spectral component of the sensor is provided by dedicated detectors that are wavelength optimized to the spectral analysis of the target gas species. The signals from the detector elements are digitized by an analog-to-digital (A/D) converter, wherein the digital signals are captured by an on-board processor, such as a microprocessor. The signals are processed to predefined computations based on stored methodology and calibration equations. The raw signals from the sensor are thus converted into component concentrations for the individual target gases. The results are transmitted out of the sensor via a defined communications interface, with a predefined communications protocol. A user can define the data format and communication mode desired based on the application of the sensor.

The interaction of the functional components, the electronics, and the detection system is important for the operation of the sensor. As previously mentioned the system is comprised of a light source, a sample enclosure or measurement chamber, an optical interface between the light source, the sample, and the detection system. The electronics of the invention integrate the light source, the detection system, and computational components—such as an onboard microprocessor for calculation of the gas composition and communications—between the sensor and the vehicle electronics.

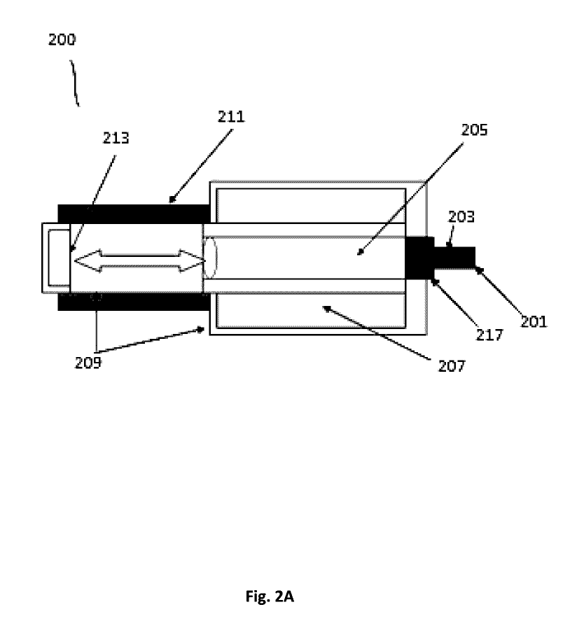

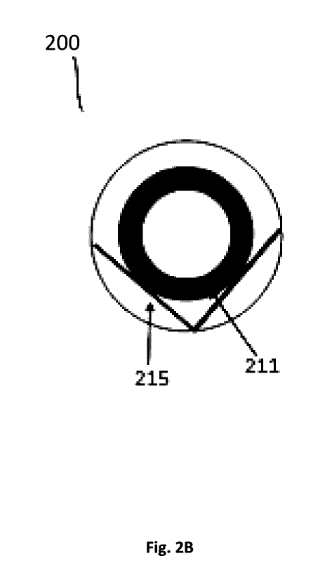

FIG. 2 illustrates an exemplary embodiment for a high temperature insertion probe 200 for the coupling of the UV-vis radiation from the light source and the sample. The insertion probe embodiment of the present invention can measure gases in situ within the flowing gas stream of an exhaust system. This insertion probe sensor comprises a light source, a two-way optical conduit, a measurement chamber that is mounted inside the exhaust gas stream, and a dedicated detector module that can measure the intensity of the light returning from the measurement chamber mounted in the gas stream.

The light source can be a pulsed xenon light source that provides wavelengths as low as about 190 nm. However, the application is intended to work from the visible range from about 720 nm (red end) to about 200 nm (the deep UV). The two way conduit can be composed from a special fiber optic or a solid light guide construction that enables light to be directed into the measurement chamber in the gas stream and to extract light returning out of the measurement chamber. The high temperature measurement chamber interface can be designed to have a retro-reflector that allows light to enter, pass through and interact with the gas stream, and then be passed back out of the measurement chamber and into the opto-electronics module of the measurement system. The high temperature interface is remote from sensitive electronics and constructed from any suitable material that enables operation in an environment up to about 800° C., and is designed to remove optical and mechanical interference from gas-borne particulate matter, such as soot.

The optimum optical path is generated between the end of an internal light guide, which can be fabricated from fused silica or quartz or any other suitable material or combination thereof, and a retro-reflecting mirror 213, as shown in FIG. 2, that can be comprised of any suitable polished metal, such as nickel or chromium. The light is transmitted from the external fiber optic coupling into the measurement chamber via the light guide. Inside the measurement chamber the light is imaged from the end of the light guide on to a reflective surface. The light reflects back to the light guide 205 and traverses back to the fiber optic interface. A two-way fiber optic, in a bifurcated format, allows light to travel to and from the retro-reflector or measurement head. The solid light guide serves as an optical coupling and a thermal insulator, providing a thermal buffer between the hot gases and the external connector on the measurement chamber. Any suitable insulating material 207 can be used for fabricating the insertion probe and sample measurement chamber, such as ceramic and stainless steel, to help prevent excessive heat within the measurement chamber and locating the light guide. The measurement chamber enclosure 209 can also use similar material, such as ceramic or stainless steel, to help prevent excessive heat.

Light returning to the detector module 217 from the retro-reflector is detected by wavelength specific detectors. The signals from these detectors can be calibrated individually and used to calculate the individual gas concentrations. The number of detector channels defines the number of different gases to be detected. To a first degree, each detector can correspond to a specific gas component. Interferences or cross-sensitivities can occur because there is not necessarily a one-to-one physical relationship between the gas components and each detector. These interferences can be calibrated and offset, and then applied to the numerical outputs for calculated gas concentrations, which are corrected in real-time to provide a more accurate assessment of the real gas concentrations. The results for exhaust gas component concentrations can be made available via a standard interface, such as the CAN bus, to the onboard vehicle/engine computer in real-time providing on-board diagnostics and control.

As shown in FIG. 2B, an external deflector shield 215 can be implemented to reduce the impact of soot on gas readings. The deflector can be mounted on the external casing 209 of the sensor and is designed to prevent the particulate matter from entering the enclosure opening. This method utilizes the dynamics of the flowing gases to divert the particulates by using a ballistic approach, which passes the gas stream over an aerodynamically shaped surface. The particulates have a mass and built-in inertia within the flowing stream, and the particulate stream may be reduced by passage over and through air vanes that deflect the particles away from the measurement aperture.

A secondary shield 211, as shown in FIG. 2A-B, in the form of a filter can also be implemented in the enclosure opening. This secondary shield 211 can be fabricated from a catalytic mesh that oxidatively degrades or combusts the soot. The secondary shield 211 can mechanically block and interact with residual particulates. The gas stream passes over and through the mesh/filter of the secondary shield 211, which may be coated with a catalytic oxidant. At the exhaust stream's elevated temperatures, the soot particles oxidize on the catalytic surface of the secondary shield 211 to gaseous carbon oxides. The secondary shield 211 is intended to operate at the elevated temperatures of the exhaust gas stream. Several different catalytic surfaces can provide this level of interaction with soot, resulting in the removal of soot from the optical chamber.

As illustrated in FIG. 2A-B, a fiber optic connector 201 can be placed on the back end of the sensor at a point of lower temperature in order to reduce the degradation of the connecter Light is transferred to and from the measurement chamber using a fiber optic cable and further interfaced to the sensor body 209 via any suitable fiber optic connector 201. Any suitable connector can be used, but one exemplary embodiment of the connector is a Sub Miniature A (SMA) connector. Connectors that are adapted for high temperature, environmentally hard conditions, or both can also be considered for use with the sensor.

Proximate to the connector 201 can be a collimator 203 to collimate the beam. This is essential in cases where the beam passage through the optical element must be optimized in terms of illumination (entrance) and beam collection (exit). If such optics are not used, there can be a large divergence angle of light from the source, and little enters a first of optical fibers, used to supply light to the sensor probe 200. Further, light returning in optical fibers to the detector also diverges over a large angle. The internal reflection measurement is highly angle dependent. Thus, in the absence of collimation optics for the source, and collection optics for the detector(s), the efficiency and optical integrity of the internal reflection device can be adversely affected, and measurement accuracy may be significantly impaired. For low-cost applications, the use of simple plastic optics can be used when fabricating the sensor.

One exemplary embodiment of the present invention has the light source, the detector, and the system electronics in a common package. In this arrangement the ideal optical interface can be a single-core or multi-core/2-way, such as a bifurcated cable. Suitable connectors are used to couple the remote sample probe to the measurement head connector and system electronics. Similarly, it is beneficial to consider the use of environmentally hardened couplings and cables or ruggedized external cable coverings to help ensure the longevity of the sensor.

Specifically, the insertion probe 200 embodiment can be used at target locations within an exhaust system for the measurement of target gases that range from the exit of the engine to the end of the tailpipe. After treatment systems are located between these two points, one of the functions of the present invention is to determine the effectiveness of the after treatment processes leading to “clean” tail pipe emissions. Another function of the sensor can include monitoring the exhaust gas composition from the engine to the end of the tailpipe for providing feedback and subsequent control of the after treatment processes based on the sensor data.

A wide range of temperatures are encountered along the length of an exhaust system and consequently the measurement head of the sensor has to be capable of operating and surviving these extreme temperatures of up to about 800° C. The key attributes of the measurement chamber are the ability to duplicate the optical interaction of a flow through system in a single ended probe where the light enters the probe from the excitation source, interacts with the target gases, and then exits and is transferred to the detection system. The only part of the system that is subjected to the high temperatures is the optical transfer system. The optical transfer system can be a retroreflective unit, such as the unit is illustrated in FIG. 2.

Both the in-line and insertion probe sensors can be used with a micro-spectrometer, but the primary focus of the present invention is the use of a measurement technology that is compact, designed for chip-scale fabrication, and mass production allowing for low cost system that is suited for a variety of markets, specifically the automotive market.

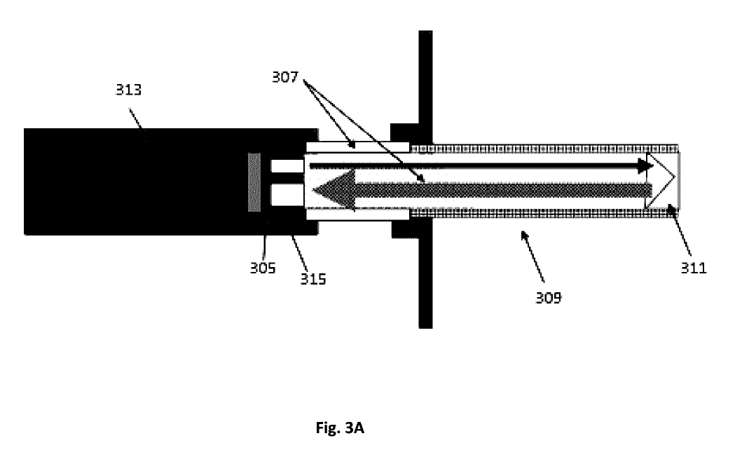

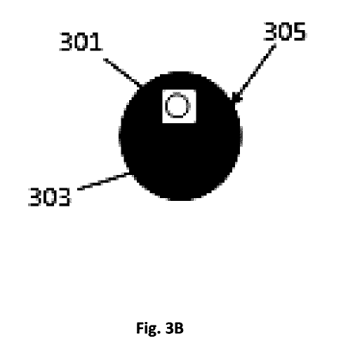

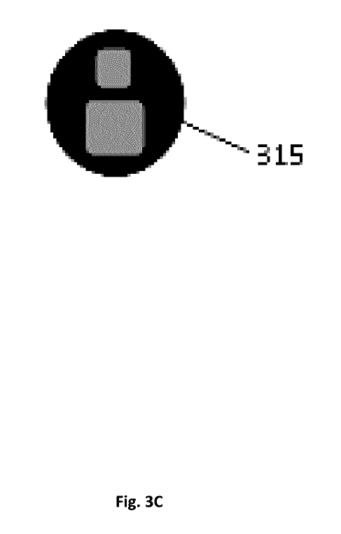

FIG. 3A-C illustrates an exemplary embodiment of a real-time measurement sensor of NOx gas species. This exemplary embodiment can use a solid state light source 301, such as an LED, and a solid state detector package, such as a standard photodiode detectors 303 for detection. The photodiode detectors 303 and light source 301 can be packaged together on an opto-board 305 within the sensor. This provides a low cost option and offers a non-species specific measurement of NOx gas in the form of NO₂. This sensor can be based on a ^{˜}400 nm LED for NO₂ and a ^{˜}700 nm LED for a reference baseline. This sensor can also be implemented with a remote insertion probe, or the LED light sources may be mounted outside the sensor enclosure and a measurement chamber 309 may be close coupled. The measurement chamber may have a light guide 307 using any suitable material, such as quartz, fused silica, or any other material or combination thereof. An optical isolator 315 can also be used to isolate the light source from the detector module, detector, or photodiode.

A coupling apparatus for coupling said solid-state source and solid-state detector to the measurement chamber can be used in the real-time measurement sensor. The measurement chamber may also include a single component optical interface fabricated as a refractive optic 311 that works in an internal reflectance or optional transmittance modes (or light scattering or fluorescence modes). Integrated electronics 313 that include circuits that provide optical compensation, temperature sensing and compensation, analog and digital signal processing, and external communications are communicatively coupled to the sensor. The system is designed to allow a high level of integration of both electronic and optical components, and to include packaging that provides both thermal isolation and ease of assembly and manufacture. Fiber optics or other forms of optical light guide or light conduit may be used, with appropriate source collimation and detector collection optical elements.

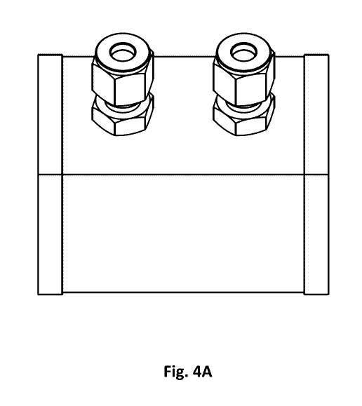

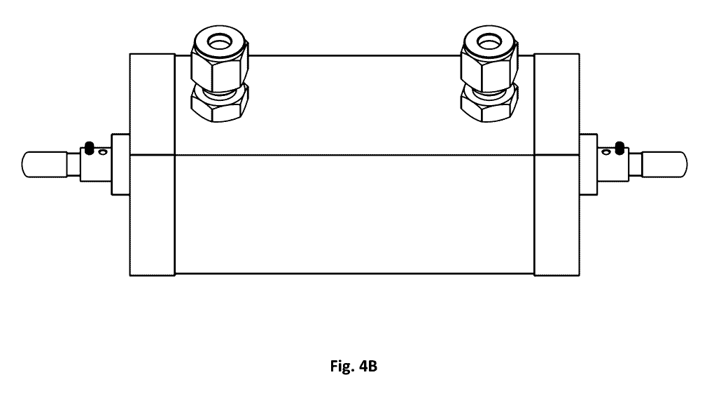

FIG. 4 illustrates two examples of measurement chambers that can be used to interface the gases to a spectrometer. The measurement chamber depicted in FIG. 4A can be fabricated from aluminum and provides about a 50 mm optical path, while FIG. 4B depicts a measurement chamber can be fabricated from stainless steel that has about a 100 mm optical path. As indicated earlier there are two practical modes of implementation flow-through and insert probe for the measurement head/chamber. The measurement chambers shown in FIG. 4 could be adapted to an onboard vehicle sensing system, but requires setting up extractive sampling.

Interfacing the sensor to an engine exhaust creates a finite limit to the optical path that can be accommodated. The maximum physical limit is about 2.5 to about 3.0 inches with regards to the physical length of the measurement chamber of the final sensor. As described earlier and illustrated in FIG. 4, the measurement chamber can range in sizes from about 50 mm to about 100 mm length. However, any suitable size that allows for the appropriate measurement of the gas can be used. The optical path length within the measurement chamber, which is two times the length of the physical path length, provides a compromise for the measurement sensitivity because of physical constraints in the mechanical length of the sensing system. With optimized signal handling this path length provides a limit of detection for the target gases in the about five parts per million (ppm) range, possibly down to about 2 ppm.

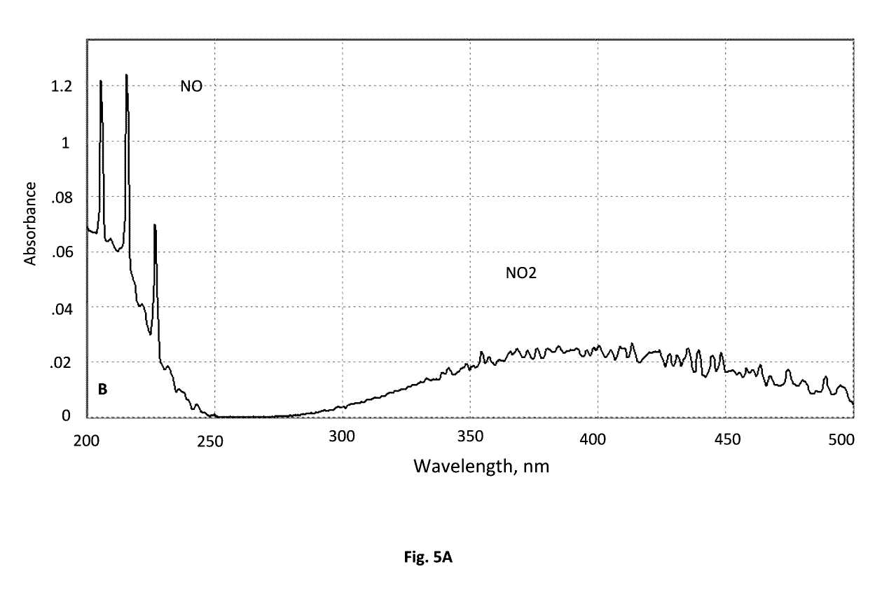

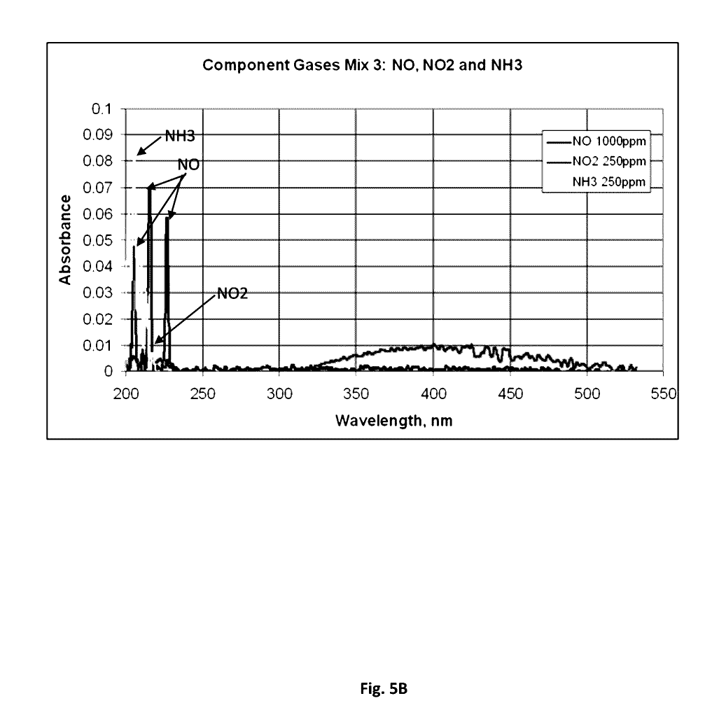

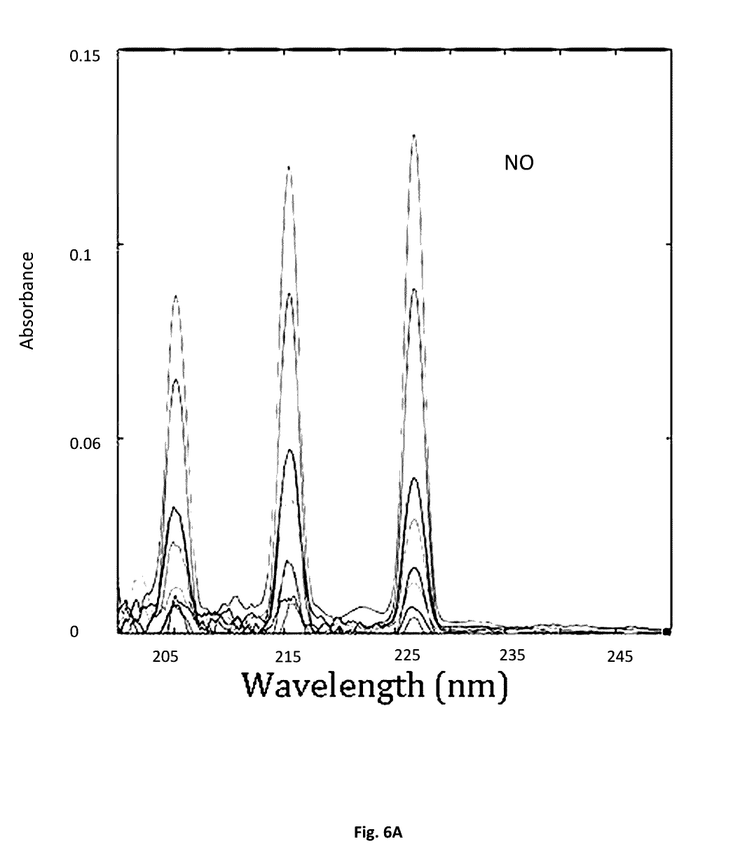

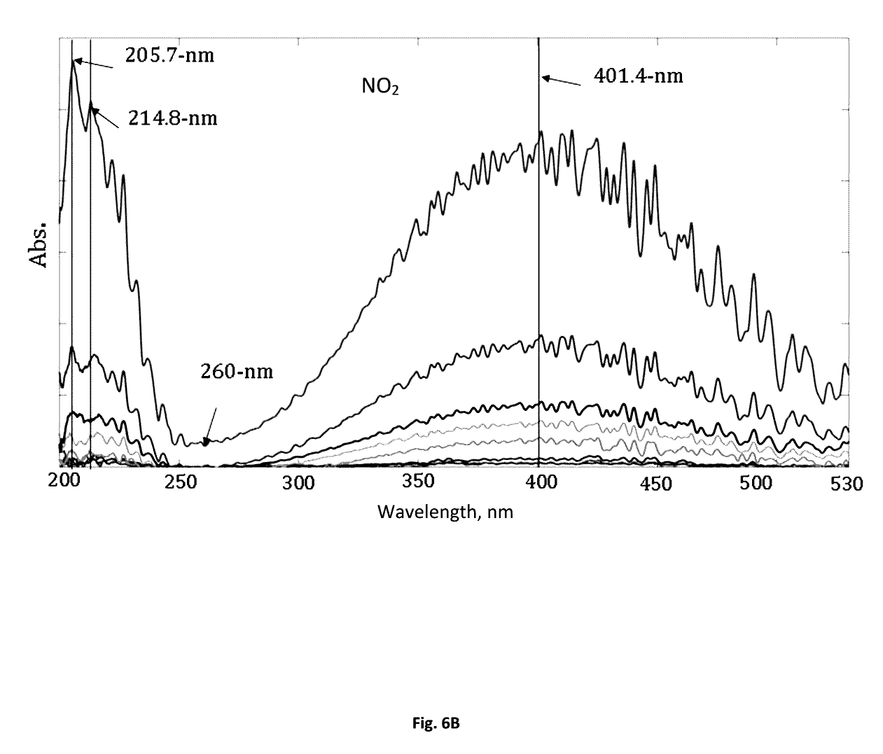

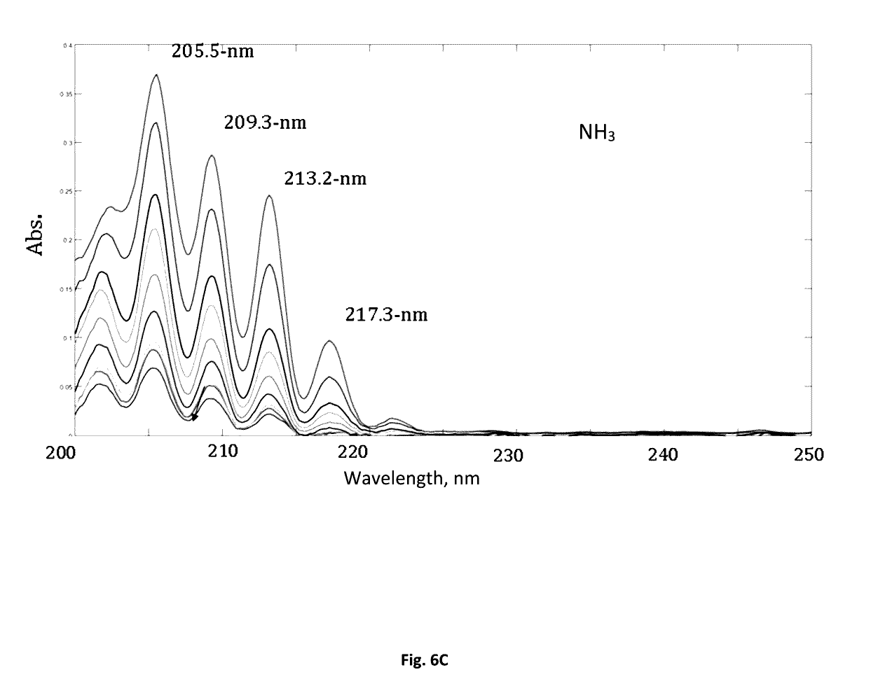

The wavelength range selected for the sensor measurement is defined as the ultraviolet extended to the visible spectrum for one NO₂ and as a baseline reference that is free from absorption from component gas species. The need to measure ammonia necessitates extending the measurement range down to about 200 nm in the deep UV, as indicated in FIGS. 5 and 6, where the ammonia absorptions are captured within a window from about 200 nm to about 220 nm. NO is the next component that requires a deep UV measurement with absorption occurring within the range from about 205 nm to about 230 nm.

SO₂ and NO₂ are measured at longer wavelengths, with absorption centers of about 287 nm and about 400 nm respectively. A reference baseline from about 650 nm to about 700 nm can be selected to ensure that this reference point is free from other absorptions. The only other absorption that may occur in the region is that of aromatic hydrocarbons, nominally centered from about 240 nm to about 260 nm. All other anticipated gas species, water vapor and carbon oxides including CO and CO₂ are transparent within the total measurement range of from about 195 nm to about 700 nm.

FIG. 5A-B illustrates the overlap of the shorter wavelength absorptions of NO and NH₃, as well as a secondary absorption of NO₂. As in many spectroscopic applications, it is necessary to apply software for deconvolution of the data for separation of the individual spectral contributions of the individual gas components. Each gas has its own unique signature, and even at low concentrations the individual gas spectra behave as they would on their own in the absence of the other gases. As a result, the spectral contributions across the spectrum for each component behave as the algebraic sum of the individual gas spectra. Within the concentration ranges considered, the relationship is either linear or can be represented by a simple second order polynomial.

Deviations from linearity are usually linked to various elements, such as unaccounted spectral contributions from one of the other components present, inadequate representation of the component gas profile, or an incorrect assessment of the reference baseline point. Additional contributions to non-linearity are increases in pressure that can cause bandwidth broadening, a wide range of temperatures, and component interactions with reactive gases. In a flowing system, with an open ended tailpipe it is anticipated that the pressure will be close to atmospheric and pressure increases will be minimal.

Gas interactions should be minimal, this is a reactive gas mixture, and some interactions between ammonia and nitrogen and sulfur dioxide might be anticipated, especially in the presence of water, and in particular at elevated temperatures. One other chemical related interaction is the interconversion of NO to NO₂ in the presence of oxygen. This can be seen in the spectrum of NO if residual oxygen/air is present in the measurement chamber or the sample path. Therefore, in a mixed gas system the individual components can be measured and can be assumed to respond linearly, or consistent with a simple polynomial. In order to account for all of the potential sources of non-linearity or interaction it is important to calibrate the system with the gases in a mixture, not as individual components. Also, it is important to monitor temperature and pressure and to be prepared to correct for temperature or pressures related perturbations.

Although spectral relationships have a linear basis it is best to assume non-linearity and to fit polynomials to the calibration curves. Even if the relationship is linear, that can be accommodated by a polynomial equation by assigning zero to the higher order coefficients. In a multicomponent system, where additional variables, such as temperature, pressure, and component interactions can occur, it is usual to build a multivariate model that includes all of the variables and covers the expect range of variance of these variables. This is accommodated in the system calibration and in the software used to compute the component concentrations. The calibration generates a series of equations that correlate with the individual variables and these are stored within the system as a series of coefficients linked to the calibration equations. In a practical system, it may be necessary to include calibration trimming equations that compensate for individual variances in the sensor responses as a function of the operating environment and unexpected extremes in the operating conditions.

What is claimed is:

1. A species-specific optical sensor device for determining properties of a sample, said device comprising:
a light source;
a sample measurement chamber having an opening for said sample;
an optical interface between said light source and the sample measurement chamber;
a detector module; and
an electronics system configured to provide energy to said device and integrates said light source, sample management chamber, and detector module.

2. The device of claim 1, further comprising a microprocessor.

3. The device of claim 2, further comprising a vehicle control system communicatively coupled to said microprocessor, wherein said vehicle control system and microprocessor communicate with each other and said vehicle control system generates a signal based on data from said microprocessor.

4. The device of claim 1, further comprising a collimator between said sample measurement chamber and said optical interface, wherein said collimator is configured to enhance measurement accuracy of said device.

5. The device of claim 1, wherein said sample measurement chamber comprises:
a light guide configured to generate an optical path of a beam emitted from said light source, and
a reflective surface, configured to reflect said beam back to said light guide and through said optical interface to said detector module.

6. The device of claim 1, further comprising an external deflector shield configured to reduce the impact of soot on readings of said sample.

7. The device of claim 1, further comprising a secondary shield, wherein said shield is a filter positioned near said opening of said sample measurement chamber, wherein said filter is configured to oxidatively degrade or combust soot or other particles.

8. The device of claim 1, wherein said light source is a light emitting diode.

9. The device of claim 1, wherein said microprocessor is configured to calculate gas compositions of said sample.

10. The device of claim 1, wherein said light source is a xenon flash lamp.

11. The device of claim 1, wherein said light source is a pulsed xenon lamp.

12. The device of claim 1 wherein said optical interface is a fiber optic cable

13. The device of claim 5, wherein said light guide is fabricated from fused silica.

14. The device of claim 5, wherein said light guide is fabricated from quartz.

15. The device of claim 1, wherein said light source emits light at a wavelength between 190 nm and 750 nm.

16. A species specific optical sensor device for determining properties of a sample, said device comprising:
a light source configured to provide a beam of light between 195 nm and 750 nm;
a detector module having at least one detector configured to detect a specific wavelength of light and transmit a correlated signal; and
a sample measurement chamber having an opening for said light source, wherein said sample measurement chamber comprises
a light guide configured to generate an optimum optical path of a beam emitted from said light source, and
a reflective surface, configured to reflect said beam back to said light guide and through said optical interface to said detector module;
an optical interface between said light source, sample measurement chamber, and detector module;
an analog-to-digital converter configured to convert said signal from said detector module,
a microprocessor capture said converted signal and process said signal; and
an electronics system configured to provide energy to said device and integrates said light source, sample management chamber, and detector module.

17. The sensor of claim 16, further comprising a secondary shield coupled to the sensor configured to block particulates from the sample measurement chamber.

18. The sensor of claim 17, wherein the secondary shield is coated with a catalytic oxidant configured to oxidize soot particulate on the surface of the secondary shield to remove soot from the sample measurement chamber.

19. The sensor of claim 18, further comprising an optical isolator configured to isolate the light source from the detector.

20. A real-time gas measurement sensor comprising:
an integrated solid-state source and solid state detector package;
a sample measurement chamber having an opening for said sample;
a coupling apparatus for coupling said integrated solid-state source and solid-state detector to said measurement chamber; and
electronics for providing energy for said source and for receiving a signal generated by said detector in response to energy coupled to said detector by said coupling apparatus, said integrated electronics providing direct output of sample properties of said sample.
